(19) United States
(12) Patent Application Publication
(10) Pub. No.: US 2020/0393440 A1
(43) Pub. Date: Dec. 17, 2020

(54) METHOD, APPARATUS AND SYSTEM FOR SINGLE-MOLECULE POLYMERASE BIOSENSOR WITH TRANSITION METAL NANOBRIDGE

(71) Applicant: Roswell Biotechnologies, Inc., San Diego, CA (US)

(72) Inventors: Sungho Jin, San Diego, CA (US); Chulmin Choi, San Diego, CA (US); Paul Mola, San Diego, CA (US); Barry Merriman, San Diego, CA (US)

(73) Assignee: Roswell Biotechnologies, Inc., San Diego, CA (US)

(21) Appl. No.: 16/840,755

(22) Filed: Apr. 6, 2020

Related U.S. Application Data
(60) Provisional application No. 62/830,231, filed on Apr. 5, 2019.

(51) Int. Cl. G01N 33/487; G01N 27/327; C12Q 1/48; C12Q 1/6869

(52) U.S. Cl. CPC G01N 33/48721; G01N 27/3271; C12Q 1/6869; C12Q 1/485; G01N 27/3275

(57) ABSTRACT

The disclosure generally relates to a method, apparatus and system for single-molecule polymerase biosensor having transition metal dichalcogenide (TMD) nanobridge for sequencing, information storage and reading. In an embodiment, the present disclosure relates to nanofabrication of biomolecular sensing devices and to the fabrication of devices for analyzing DNA and related biomolecules. In still another embodiment, the disclosure relates to a DNA-based memory system.

This application claims priority to, and the benefit of, U.S. Provisional Patent Application Ser. No. 62/830,231, filed on Apr. 5, 2019, and entitled “SINGLE-MOLECULE POLYMERASE BIOSENSOR COMPRISING TRANSITION METAL DICHALCOGENIDE NANOBRIDGE FOR SEQUENCING, INFORMATION STORAGE AND READING,” the contents of which are incorporated by reference in their entirety.

### FIELD

The disclosure relates to method, apparatus and system for single-molecule polymerase biosensor having transition metal dichalcogenide nanobridge for sequencing, information storage and reading. In an embodiment, the present disclosure relates to nanofabrication of biomolecular sensing devices and to the fabrication of devices for analyzing DNA and related biomolecules. In still another embodiment, the disclosure relates to a DNA-based memory system.

### BACKGROUND

Analysis of biomolecules including DNAs and genomes has received an increasing amount of attention in recent years in various fields of precision medicine or nanotechnology. The seminal work of Maclyn McCarty and Oswald T. Avery in 1946, (see, “Studies On The Chemical Nature Of The Substance Inducing Transformation Of Pneumococcal Types II. Effect Of Desoxyribonuclease On The Biological Activity Of The Transforming Substance,” The Journal of Experimental Medicine 83(2), 89-96 (1946)), demonstrated that DNA was the material that determined traits of an organism. The molecular structure of DNA was then first described by James D. Watson and Francis HC Crick in 1953, (see a published article, “Molecular structure of nucleic acids.”, Nature 171,737-738 (1953)), for which they received the 1962 Nobel Prize in Medicine. This work made it clear that the sequence of chemical letters (bases) of the DNA molecules encode the fundamental biological information. Since this discovery, there has been a concerted effort to develop means to actually experimentally measure this sequence. The first method for systematically sequencing DNA was introduced by Sanger, et al in 1978, for which he received the 1980 Nobel Prize in Chemistry. See an article, Sanger, Frederick, et al., “The nucleotide sequence of bacteriophage φX174.” Journal of molecular biology 125, 225-246 (1978).

Sequencing techniques for genome analysis evolved into utilizing automated commercial instrument platform in the late 1980's, which ultimately enabled the sequencing of the first human genome in 2001. This was the result of a massive public and private effort taking over a decade, at a cost of billions of dollars, and relying on the output of thousands of dedicated DNA sequencing instruments. The success of this effort motivated the development of a number of “massively parallel” sequencing platforms with the goal of dramatically reducing the cost and time required to sequence a human genome. Such massively parallel sequencing platforms generally rely on processing millions to billions of sequencing reactions at the same time in highly miniaturized microfluidic formats. The first of these was invented and commercialized by Jonathan M. Rothberg's group in 2005 as the 454 platform, which achieved thousand fold reductions in cost and instrument time. See, an article by Marcel Margulies, et al., “Genome Sequencing in Open Microfabricated High Density Picoliter Reactors,” Nature 437, 376-380 (2005). However, the 454 platform still required approximately a million dollars and took over a month to sequence a genome.

The 454 platform was followed by a variety of other related techniques and commercial platforms. See, articles by M. L. Metzker, “Sequencing Technologies—the Next Generation,” Nature reviews genetics 11(1), 31-46 (2010), and by C. W. Fuller et. al, “The Challenges of Sequencing by Synthesis,” Nature biotechnology 27(11), 1013-1023 (2009). This progress lead to the realization of the long-sought “$1,000 genome” in 2014, in which the cost of sequencing a human genome at a service lab was reduced to approximately $1,000, and could be performed in several days. However, the highly sophisticated instrument for this sequencing cost nearly one million dollars, and the data was in the form of billions of short reads of approximately 100 bases in length. The billions of short reads often further contained errors so the data required interpretation relative to a standard reference genome with each base being sequenced multiple times to assess a new individual genome.

Thus, further improvements in quality and accuracy of sequencing, as well as reductions in cost and time are still needed. This is especially true to make genome sequencing practical for widespread use in precision medicine (see the aforementioned article by Fuller et al), where it is desirable to sequence the genomes of millions of individuals with a clinical grade of quality.

While many DNA sequencing techniques utilize optical means with fluorescence reporters, such methods can be cumbersome, slow in detection speed, and difficult to mass produce to further reduce costs. Label-free DNA or genome sequencing approaches provide advantages of not having to use fluorescent type labeling processes and associated optical systems, especially when combined with electronic signal detection that can be achieved rapidly and in an inexpensive way.

In this regard, certain types of molecular electronic devices can detect single molecule, biomolecular analytes such as DNAs, RNAs, proteins, and nucleotides by measuring electronic signal changes when the analyte molecule is attached to a circuit, for example, a field effect transistor device. Such methods are label-free and thus avoid using complicated, bulky and expensive fluorescent type labeling apparatus. These methods can be useful for lower cost sequencing analysis of DNA, RNA and genome.

Certain types of molecular electronic devices can detect the biomolecular analytes such as DNAs, RNAs, proteins, and nucleotides by measuring electronic signal changes when the analyte molecule is attached to the circuit comprising a pair of conductive electrodes. Such methods are label-free and thus avoids using complicated, bulky and expensive fluorescent type labeling apparatus. One example of sequencing-by-synthesis approach is to utilize a single molecule polymerase with incorporated DNAs, the sequence of which is detected through a current pulse signal when each type of the nucleotides (A, T, C, G) is attached to the polymerase complex with a distinct electrical signal.

While current molecular electronic devices can electronically measure molecules for various applications, they lack the reproducibility as well as scalability and manufacturability needed for rapidly sensing many analytes at a scale of up to millions in a practical manner. Such highly scalable methods are particularly important for DNA sequencing applications, which often need to analyze millions to billions of independent DNA molecules. In addition, the manufacture of current molecular electronic devices is generally costly due to the high level of precision needed.

### SUMMARY

Disclosed herein are principles that provide new and improved sequencing apparatuses, structures and methods using two-dimensional layer structured semiconductors, which can provide reliable DNA genome analysis performance and are amenable to scalable manufacturing.

Two dimensional (2D) layered transition metal dichalcogenides (TMDs) materials and devices have attracted a great deal of interest due to their novel electronic, physical and chemical characteristics. One example is MoS₂ which can be incorporated as a sensor device. MoS₂ type 2D materials can be a single layered material or several layered material, and can be obtained by various techniques, such as e.g., by isolation of very thin MoS₂ layer through mechanical exfoliation, physical or chemical vapor deposition, molecular beam epitaxy (MBE) type construction or sulfurization of transition metal layer such as Mo or W.

Transition metal dichalcogenide (TMD) monolayers are in general atomically thin semiconductors of the type MX₂, which M a transition metal atom (notably Mo, W, or Ti, Zr, Hf, V, Nb, Ta, Tc, Re, Co, Rh, Ir, Ni, Pd, Pt) and X a chalcogen atom (S, Se, or Te). One layer of M atoms is sandwiched between two layers of X atoms. A MoS₂ monolayer can be about 6.5 Å thick. TMD monolayers of e.g., MoS₂, WS₂, MoSe₂, WSe₂, MoTe₂ have a direct band gap, and can be used in electronics as transistors or sensors. Either monolayer TMD or few layer TMD can be structurally modified, to be utilized as solid state DNA or genome sensors. Being an ultrathin direct bandgap semiconductor, a transition metal dichalcogenide such as single-layer MoS₂ may have potential for widespread applications in nanoelectronics, optoelectronics, and energy harvesting.

The layered TMDs typically have a hexagonal type structure with space group P63/mmc. It should be noted that monolayers of TMD materials are not just one atom thick as graphene, but are made up with tri-atomic thick layers consisting of metal atoms (such as Mo or W) sandwiched between two layers of chalcogen atoms (such as S, Se, or Te). The atoms in-plane in MoS₂ type 2D materials are put together and bonded by strong covalent bonds. The adjacent layers of TMD like MoS₂ along the thickness direction are joined together by a weak van der Wall force binding. This force is strong enough to hold the layers together with mechanical integrity. The TMD materials provide interesting and unique possibilities to design electronic devices involving hetero structures. The direct band gap of TMD monolayers is tunable with the application of the mechanical strain.

Single nucleotide identification and DNA sequencing have already been demonstrated with biological nanopores or solid state nanopores such as those in graphene and MoS₂ layers. A DNA type molecule is threaded through a nanopore under an applied electric field, so that the sequence of nucleotides is read by monitoring small changes in the ionic current flowing through the pore, which are induced by individual nucleotides temporarily residing within the pore during threading. However, the fragility of such pores, together with difficulties related to reproducible and low noise measurement of detection signals in nanopore sequencing methods in general are some of the current issues that need to be addressed.

The disclosed principles provide, among others, new biomolecular sensor devices and associated methods, employing transition metal dichalcogenide nanoribbons as a component of molecular bridge, which in turn comprises an attached, preferably single molecule polymerase to analyze DNA lines or fragments by step by step attachments of nucleotides or short DNA fragments.

Various embodiments are disclosed herein regarding specially processed, 2D layer-containing enzyme polymerase sensor device structures and methods of manufacture for a multitude of devices for use in electronic DNA, RNA or genome sequencing systems. Unique geometrical modifications are made so as to enable a construction of sensor device comprising only a single molecule polymerase enzyme for more accurate electronic analysis. Such label-free, single molecule based sequencing analysis systems utilize preferably a nanoscale dimension-controlled, transition metal dichalcogenide (TMD) micro-ribbon or nano-ribbon bridge. The electronic system may also be used in analyzing other types of biomolecules, such as proteins, depending on how the molecular sensors are functionalized to interact with biomolecule sensing targets. The TMD-based sequencing systems disclosed here can be assembled into a massively parallel configuration for rapid analysis of targets including nucleotides, in particular for applications to sequencing of a DNA molecule, or a collection of such molecules constituting an entire human genome. Such systems in the present disclosure can also be used for DNA-based information storage, for example, for archival storage of huge volume of information in human society.

### BRIEF DESCRIPTION OF THE DRAWINGS

The features and advantages of the embodiments of the present disclosure will become more apparent from the detailed description set forth below when taken in conjunction with the drawings. The drawings and the associated descriptions are provided to illustrate embodiments of the disclosure and not to limit the scope of what is claimed.

FIGS. 1A and 1B. (Design option 1)—Side view for fabrication steps for MoS₂ ribbon bridge based biosensor, with size-limited, MoS₂ or TMD or semiconductor islands exposed, with optionally defective MoS₂, and any damage by nanopatterning repaired by annealing or plasma-treatment. (a) deposit and pattern conducting electrode pair (source and drain) and place a suspended MoS₂ layer, (b) To enable Nanoimprinting, planarize the surface, then use vertical Au nanopillar, and use size-limited MoS₂ region, (c) allow preferably a single molecule enzyme (DNA, RNA polymerase, etc) to be attach on exposed MoS₂, (using biotin-streptavidin, antigen-antibody, peptide complex, etc) for polymerase reaction of nucleotide attachment for FET sequencing, or other protein sensing.

FIGS. 2A and 2B. (Design option 2)—Exemplary design of single molecule bridge of DNA or RNA sensor comprising size-limited, MoS₂ type 2D chalcogenide semiconductor nano-ribbon region. The polymerase reaction of nucleotide attachment (nucleotide monomers like A, T, C, G, etc) alters the electrical current properties of the FET molecular bridge on pulsing for sequencing analysis.

FIGS. 3A and 3B. (Design option #3)—Alternative design using temporary, removable guiding channel to allow a single polymerase attachment on MoS₂ nanoribbon bridge, but can be dissolved away later. (a) Removable tapered channel prepared by microfabrication or nanoimprinting, (b) Guided single polymerase attachment onto MoS₂ nanoribbon bridge, followed by dissolution removal of the sacrificial guide structure above.

FIGS. 4A and 4B schematically illustrates a cross-sectional view of producing diameter-reduced-tip Au electrode for size-confined molecular bridge sensor formation.

FIGS. 5A and 5B schematically sectional view showing an example process of using a sacrificial plug to create a 5-10 nm, size-limited structure to place a single molecule polymerase sensor. Either nanoimprinting or sequential multilayer resists having gradually different dissolution rate to create a funnel shape type guidance geometry.

FIG. 6 provides an alternative method of guidance funnel shaping using nanoimprinting for 5-10 nm regime, in the PMMA or HSQ type resist to enable a placement of a single molecule polymerase sensor on MoS₂ nanoribbon bridge. The protruding PMMA structure above also protects the attached polymerase from mechanically washed away by microfluidic solution flow or post-sequencing washing operation.

FIG. 7 illustrates use of additional force parameters to assist in guidance and placement of single polymerase or single biosensor molecule per each of the electrode array structure.

FIGS. 8A and 8B (Design alternative 4)—Exemplary design of utilizing size-confined DNA assembly well to have only a “Single Streptavidin” immobilized for sequencing or protein sensing.

FIG. 9. (Design alternative 5)—Cross-sectional view of example design of utilizing shape memory type metal, ceramic or polymer functional material for temperature-, magnetic-, E-field-, pH- or chemical-responsive, dimension-changeable structure to physically trap only a “Single Streptavidin” or “single Polymerase” for high conductivity sequencing or protein sensing.

FIG. 10. Top view of an array of TMD bridges like MoS2 or WS2, bridge molecular sensors, with a size-limiting structure for single polymerase. Massively parallel electronic sequencing analysis can be performed with many devices organized into a system, having as many as 10,000 or even at least 1 million devices.

FIG. 11. MoS₂ ribbon transfer deposited. A large sheet MoS₂ (e.g., >1 cm²) can be transfer deposited on CMOS chip or other device surface, and e-beam or nano-imprint patterned. For less time-consuming process/assembly, pre-slitted (but still frame-attached) MoS₂ could be transferred, or an array of nano-ribbons can be wet-stamp transferred onto the device surface, preferably using an elastomeric stamp (e.g., PDMS based), with optional binding-assisting thin layer of polymer type material, which can be later washed or burned away.

FIGS. 12A and 12B. MoS₂ nano-ribbon array patterned on flat SiO₂ or other removable substrate surface, to be made transferable and releasable by a sequence of processes.

FIG. 13. Transfer of MoS₂ nanoribbons by PDMS type soft stamp. The PDMS stamp optionally has protruding ridges for easier pick up of the nanoribbons.

FIG. 14. Top view of an array of Au electrode pairs (or other metal) with float-transferred or PDMS stamp-transferred MoS₂ bridges, with redundant ribbons to ensure at least one bridge formation occurs. Optionally, a masking coating with blocking agent may be added to prevent biotin, streptavidin or polymerase adhesion, except for a local ˜5 nm circle on MoS₂ surface, so as to ensure only a single enzyme molecule is attached.

FIG. 15. (a) Tethered array of encoded (memory written) DNA fragments periodically positioned on a substrate, (b) Polymerase-MoS₂ nanobridge array approaching DNA array being released, (c) DNA templates are taken up by polymerase array and the completed sensor array is moved array for DNA sequencing to read the recorded memory information. (Microfluidics chamber not shown). Massive parallel DNA written information array in combination with massively parallel MoS₂ nanobridge reader array allows ultrafast, “random-access-enabled” DNA memory retrieval. Several different configuration of tethered DNA memory array, with various methods of tethering and releasing, can be made to maximize the DNA memory processing capability.

The drawings are illustrative of the disclosed principles and not limiting thereof.

### DETAILED DESCRIPTION

The disclosed embodiments generally provide a sequencing apparatus, structures, and methods for using two-dimensional, layer structured semiconductors to provide DNA and genome analysis performance. The disclosed embodiments are amenable to scalable manufacturing.

Two dimensional (2D) layered materials such as transition metal dichalcogenides (TMDs) materials and devices have received much attention in recent years by virtue of their unique electronic, physical and chemical properties. One example is molybdenum dichalcogenide MoS₂ which can be incorporated as a sensor device. MoS₂ type 2D materials can be a single layered material or several layered material. The 2D layer materials such as MoS₂ can be produced by various known techniques, e.g., by isolation of very thin MoS₂ layer through mechanical exfoliation, physical or chemical vapor deposition, molecular beam epitaxy (MBE) type construction, or sulfurization of a transition metal layer such as Mo or W.

### Definitions

As used herein, “Nucleotide” means either the native dNTPs like A, T, C, G (i.e., dATP, dTTP, dCTP and dGTP), or collectively refers to various types of modified dNTPs as described above.

As used herein, “Polymerase” means an enzyme that synthesizes long chains or polymers of nucleic acids. For example, DNA polymerase and RNA polymerase can copy a DNA or RNA template strand, respectively, using base-pairing interactions, which is utilized to assemble DNA and RNA molecules.

### TMD Layers and Combined TMD Materials for Sensor Bridges

In some embodiments, a TMD layer is incorporated as a part of sensor bridge structure to attach an enzyme type biomolecule to attract various types of nucleotides for electronic detection signals.

Two dimensional transition metal dichalcogenide (TMD) monolayers are in general atomically thin semiconductors of the type MX₂, with M a transition metal atom (notably including Mo, W, or Ti, Zr, Hf, V, Nb, Ta, Tc, Re, Co, Rh, Ir, Ni, Pd, Pt) and X a chalcogen atom (such as S, Se, or Te.). One layer of M atoms is sandwiched between two layers of X atoms. Both the transition metal and the chalcogenide element can be partly replaced (or doped) with other elements. Therefore, the two dimensional TMD layer incorporated into the molecular sensor bridge construction can have various modified or altered composition ranges, including the following:

(i) MoS₂, WS₂, or TiS₂, ZrS₂, HfS₂, VS₂, NbS₂, TaS₂, TcS₂, ReS₂, CoS₂, RhS₂, IrS₂, NiS₂, PdS₂, PtS₂ and their modifications or combinations, including modified stoichiometry of sulfur contents having MX₍₂₋ₓ₎ or MX₍₂₊ₓ₎ with the desired value of x in the range of 0-1.0, preferably in the range of 0-0.5, even more preferably 0-0.3. For some embodiments, the sulfur stoichiometry is intentionally altered so as to provide vacancy defects, interstitial defects, and aggregated defects so as to increase the surface energy and enhance the adhesion of biomolecule to the bridge sensor. Such defects can also increase the bandgap for stronger sensor signals;

(ii) MoSe₂, WSe₂, or TiSe₂, ZrSe₂, HfSe₂, VSe₂, NbSe₂, TaSe₂, TcSe₂, ReSe₂, CoSe₂, RhSe₂, IrSe₂, NiSe₂, PdSe₂, PtSe₂ and their modifications or combinations, including modified stoichiometry of selenium contents having MX₍₂₋ₓ₎ or MX₍₂₊ₓ₎ with the desired value of x in the range of 0-1.0, preferably in the range of 0-0.5, even more preferably 0-0.3. For some embodiments, the selenium stoichiometry is intentionally altered so as to provide vacancy defects, interstitial defects, and aggregated defects so as to increase the surface energy and enhance the adhesion of biomolecule to the bridge sensor. Such defects can also increase the bandgap for stronger sensor signals;

(iii) MoTe₂, WTe₂, or TiTe₂, ZrTe₂, HfTe₂, VTe₂, NbTe₂, TaTe₂, TcTe₂, ReTe₂, CoTe₂, RhTe₂, IrTe₂, NiTe₂, PdTe₂, PtTe₂ and their modifications or combinations, including modified stoichiometry of tellurium contents having MX₍₂₋ₓ₎ or MX₍₂₊ₓ₎ with the desired value of x in the range of 0-1.0, preferably in the range of 0-0.5, even more preferably 0-0.3. For some embodiments, the tellurium stoichiometry is intentionally altered so as to provide vacancy defects, interstitial defects, and aggregated defects so as to increase the surface energy and enhance the adhesion of biomolecule to the bridge sensor. Such defects can also increase the bandgap for stronger sensor signals;

(iv) Mixed TMD compounds in which the MX₂ compound has mixed metals and/or mixed chalcogenide. For example Mo(SₓSe_{y}Te_{z})₂, W(SₓSe_{y}Te_{z})₂, or Ti(SₓSe_{y}Te_{z})₂, Zr(SₓSe_{y}Te_{z})₂, Hf(SₓSe_{y}Te_{z})₂, V(SₓSe_{y}Te_{z})₂, Nb(SₓSe_{y}Te_{z})₂, Ta(SₓSe_{y}Te_{z})₂, Tc(SₓSe_{y}Te_{z})₂, Re(SₓSe_{y}Te_{z})₂, Co(SₓSe_{y}Te_{z})₂, Rh(SₓSe_{y}Te_{z})₂, Ir(SₓSe_{y}Te_{z})₂, Ni(SₓSe_{y}Te_{z})₂, Pd(SₓSe_{y}Te_{z})₂, Pt(SₓSe_{y}Te_{z})₂ where the combined (x+y+z) is 1-3, preferably 0.5-1.5, even more preferably 0.7-1.3. Alternatively two or more metals can be combined for sulfur containing, Se-containing or Te-containing TMD layers, e.g., (MoₓW_{y}Co_{z})S₂, (HfₓW_{y}Co_{z})Te₂ and so forth; or

(v) M_{(1-w)}N_{y}X_{(2-z)}Y_{z} structure in which the transition metal M is partially substituted with non-transition elements N, with a concentration of w and the N element selected from one or more of Al, Si, Ga, Ge, In, Sn, Sb, Bi, Al, Na, K, Ca, Mg, Sr, Ba, with the w value in the range of 0-0.3, and the chalcogenide element X partially substituted with a non-chalcogenide element Y, with the Y element selected from one or more of Li, B, C, N, O, P, F, Cl, I, with the z value in the range of 0-0.3.

In some embodiments, the thickness of a MoS₂ monolayer can be about 6.5 Å. The TMD materials in their simplest monolayer structure, e.g., MoS₂, WS₂, MoSe₂, WSe₂, MoTe₂, have a direct band gap, and hence can be used in electronics as transistors or sensors. Either monolayer TMD or few layer TMD can be structurally modified, to be utilized as solid state DNA or genome sensors, without labeling with optical capability. Being an ultrathin direct bandgap semiconductor, a transition metal dichalcogenide such as single-layer MoS₂ has found some useful applications in nanoelectronics, optoelectronics, and energy harvesting. However, not many sensor applications have been attempted or demonstrated with proper characteristics, especially for DNA or genome sequencing purposes.

Disclosed herein are label-free DNA or RNA sequencing device structures utilizing a TMD-based frame with an enzyme polymerase for detection of electronic signals when an individual nucleotide is attached onto a nucleic acid template. In some embodiments, two dimensional semiconductors of processed, defective or nanoporous Transition Metal Dichalcogenide (TMD) layer material are employed so as to utilize altered bandgaps of the TMD layer and enhanced attachment of single biomolecules. In some embodiments, the TMD-based sequencing systems invented here can be assembled into a massively parallel configuration for rapid analysis of targets including nucleotides, in particular for applications of sequencing of a DNA molecule, or a collection of such molecules constituting an entire genome. Such systems are also useful for DNA-based information storage, for which the writing is performed by encoding specific nucleotide-based arrangements or sequences and the reading is carried out by sequencing analysis using TMD-bridge based molecular sensor array.

### Compositions

In some embodiments, a sequencing device is provided comprising: (a) an electrode array of conducting electrode pairs, each pair of electrodes comprising a source and a drain electrode separated by a nanogap, said electrode array deposited and patterned on a dielectric substrate, and optionally comprising a third electrode as a gated system; (b) a single-layer or few-layer thick transition metal dichalcogenide (TMD) layer disposed on each pair of electrodes, connecting each source and drain electrode within each pair, and bridging each nanogap of each pair; (c) a dielectric masking layer disposed on the TMD layer and comprising size-limited openings that define exposed TMD regions, each opening sized so as to allow only a single enzyme biomolecule to fit therein and to attach onto the exposed TMD region defined by each opening; (d) an enzyme molecule attached to each exposed TMD region such that only one enzyme molecule is found within each opening; and (d) a microfluidic system encasing the electrode array, wherein attachment or detachment of a biomolecule selected from the group consisting of a nucleotide monomer, a protein, and a DNA segment, onto the enzyme molecule, one at a time, can be monitored as a uniquely identifiable electrical signal pulse to determine the specific nature of the biomolecule attaching or detaching.

In some embodiments, the dielectric substrate comprises SiO2. In some embodiments, the dielectric substrate comprises SiO2 or Al2O3.

In some embodiments, the TMD is selected from MoS2, WS2, TiS2, ZrS2, HfS2, VS2, NbS2, TaS2, TcS2, ReS2, CoS2, RhS2, IrS2, NiS2, PdS2, PtS2 and their modifications or combinations. In some embodiments, the TMD is MoS₂. In some embodiments, the TMD is WS₂

In some embodiments, the TMD is selected from MoS2, WS2, TiS2, ZrS2, HfS2, VS2, NbS2, TaS2, TcS2, ReS2, CoS2, RhS2, IrS2, NiS2, PdS2, PtS2 and their modifications or combinations, including modified stoichiometry of sulfur contents having MX(2−x) or MX(2+x) wherein xis in the range of 0-1.0, preferably in the range of 0-0.5, even more preferably 0-0.3. In some embodiments, the TMD is selected from MoS₂, WS₂, TiS₂, ZrS₂, HfS₂, VS₂, NbS₂, TaS₂, TcS₂, ReS₂, CoS₂, RhS₂, IrS₂, NiS₂, PdS₂, PtS₂ and their modifications or combinations and the stoichiometry of sulfur is not modified.

In some embodiments, the TMD is selected from MoSe₂, WSe₂, or TiSe₂, ZrSe₂, HfSe₂, VSe₂, NbSe₂, TaSe₂, TcSe₂, ReSe₂, CoSe₂, RhSe₂, IrSe₂, NiSe₂, PdSe₂, PtSe₂ and their modifications or combinations, including modified stoichiometry of selenium contents having MX₍₂₋ₓ₎ or MX₍₂₊ₓ₎ with the desired value of x in the range of 0-1.0, preferably in the range of 0-0.5, even more preferably 0-0.3. In some embodiments, the TMD is selected from MoSe₂, WSe₂, or TiSe₂, ZrSe₂, HfSe₂, VSe₂, NbSe₂, TaSe₂, TcSe₂, ReSe₂, CoSe₂, RhSe₂, IrSe₂, NiSe₂, PdSe₂, PtSe₂ and their modifications or combinations and the stoichiometry of selenium is not modified.

In some embodiments, defects are artificially introduced into TMD. In some embodiments, the defects are introduced to increase bandgap. In some embodiments, the defects are introduced to provide active site edge locations for strong adhesion of bridge structures or biomolecules such as enzyme molecules.

In some embodiments, TMD is selected from MoTe₂, WTe₂, or TiTe₂, ZrTe₂, Hffe₂, VTe₂, NbTe₂, TaTe₂, TcTe₂, ReTe₂, CoTe₂, RhTe2, IrTe2, NiTe2, PdTe2, PtTe2 and their modifications or combinations.

In some embodiments, TMD is selected from MoTe2, WTe2, or TiTe2, ZrTe2, HfTe2, VTe2, NbTe2, TaTe2, TcTe2, ReTe2, CoTe2, RhTe2, IrTe2, NiTe2, PdTe2, PtTe2 and their modifications or combinations, including modified stoichiometry of Tellurium contents having MX(2−x) or MX(2+x) with the desired value of x in the range of 0-1.0, preferably in the range of 0-0.5, even more preferably 0-0.3. In some embodiments, TMD is selected from MoTe2, WTe2, or TiTe2, ZrTe2, HfTe2, VTe2, NbTe2, TaTe2, TcTe2, ReTe2, CoTe2, RhTe2, IrTe2, NiTe2, PdTe2, PtTe2 and their modifications or combinations and the stoichiometry of tellurium is not modified.

In some embodiments, the tellurium stoichiometry is intentionally altered so as to provide vacancy defects, interstitial defects, and aggregated defects in order to increase the surface energy of the TMD layer and enhance the adhesion of biomolecule to the bridge sensor for stronger sensor signals.

In some embodiments, the TMD comprises a mixed TMD selected from TMD compounds in which the MX2 compound has mixed metals and/or mixed chalcogenide, selected from the group consisting of Mo(SxSeyTez)2, W(SxSeyTez)₂, Ti(SₓSe_{y}Te_{z})₂, Zr(SₓSe_{y}Te_{z})₂, Hf(SₓSe_{y}Te_{z})₂, V(SₓSe_{y}Te_{z})₂, Nb(SₓSe_{y}Te_{z})₂, Ta(SₓSe_{y}Te_{z})₂, Tc(SₓSe_{y}Te_{z})₂, Re(SₓSe_{y}Te_{z})₂, Co(SₓSe_{y}Te_{z})₂, Rh(SₓSe_{y}Te_{z})₂, Ir(SₓSe_{y}Te_{z})₂, Ni(SₓSe_{y}Te_{z})₂, Pd(SₓSe_{y}Te_{z})₂, and Pt(SₓSe_{y}Te_{z})₂ wherein the combined (x+y+z) is 1-3, 0.5-1.5, or 0.7-1.3.

In some embodiments, two or more metals are combined for sulfur containing, Se-containing or Te-containing TMD layers.

In some embodiments, the TMD layer comprises MoₓW_{y}Co_{z})S₂ or (HfₓW_{y}Co_{z})Te₂.

In some embodiments, the TMD comprises a M(1−w)NφX(2−z)Yz structure in which the transition metal M is partially substituted with non-transition elements N, with a concentration of w and the N element selected from one or more of Al, Si, Ga, Ge, In, Sn, Sb, Bi, Al, Na, K, Ca, Mg, Sr, Ba, with the w value in the range of 0-0.3, and the chalcogenide element X partially substituted with a non-chalcogenide element Y, with the Y element selected from one or more of Li, B, C, N, O, P, F, Cl, I, with the z value in the range of 0-0.3. In some embodiments, the w value is greater than 0.3, for example, greater than 0.4, greater than 0.5, greater than 0.6, greater than 0.7, greater than 0.8, greater than 0.9, or greater than 1.0. In some embodiments, the z value is greater than 0.3, for example, greater than 0.4, greater than 0.5, greater than 0.6, greater than 0.7, greater than 0.8, greater than 0.9, or greater than 1.0.

In some embodiments, the metallic conducting electrode pair is selected from Au, Pt, Ag, Pd, Rh, Ru, or their alloys.

In some embodiments, the nanogap is 5-20 nm. In some embodiments, the nanogap is less than 5 nm, for example less than 3 nm, less than 1.0 nm. In some embodiments, the nanogap is greater than 20 nm, for example, greater than 25 nm, greater than 30 nm, greater than 35 nm, greater than 40 nm, greater than 45 nm, or greater than 50 nm.

In some embodiments, the size-limiting openings are preferably less than 30 nm average equivalent diameter each, more preferably less than 20 nm equivalent diameter, even more preferably less than 10 nm equivalent diameter, by lithographically or nanofabrication defined coverage of dielectric material layer of polymer or ceramic outside a specific region intended for attaching only a single molecule. Polymerase molecules as well as streptavidin type link molecules often have a dimension on the order of −5 nm regime.

Various aspects of the invention items including the biosensor structures, materials, geometries, as well as fabrication methods and application methods are described below.

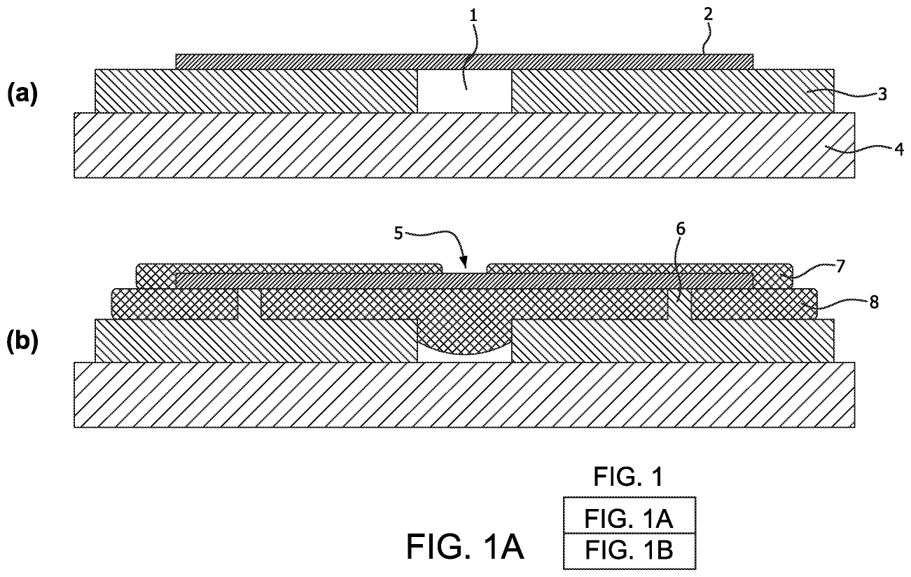

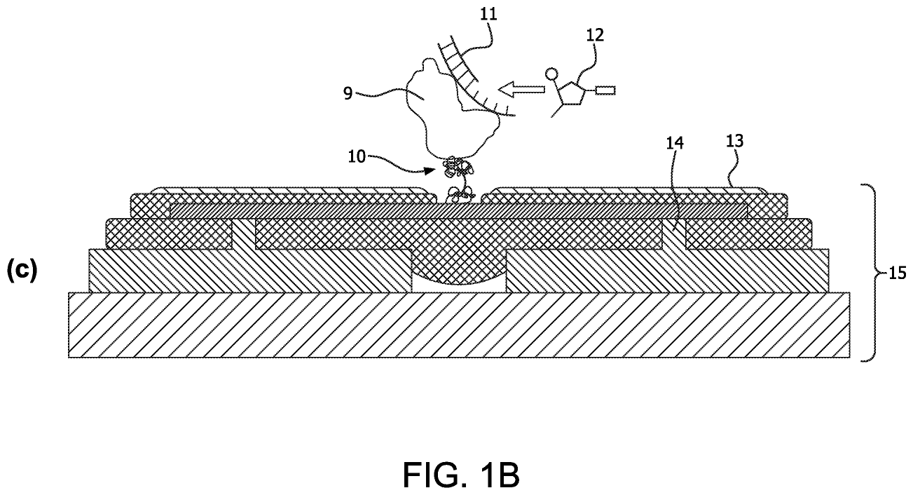

With reference now to FIGS. 1A and 1B, FIG. 1A(a) shows “Nanogap MoS₂ sequencing,” a conducting electrode pair 3 (Au, Pt, Pd, Ru, Rh, or alloys) with a nanogap 1 spacing (2-20 nm), on a dielectric substrate 4 (SiO₂, etc., optionally on Si). FIG. 1A (a) also shows a suspended MoS₂ layer 2 (or TMD transition metal dichalcogenide layer, or other semiconductor bridge), having a rectangular, or narrow-ribbon configuration, nanopatterned by nanoimprint, e-beam litho, or stamp transfer.

FIG. 1A(b) shows “Nanogap MoS₂ with Au pillars,” a size-limited (e.g., 5 nm dia.), exposed MoS₂ region 5 for preferably a single biomolecule (e.g., polymerase enzyme) attachment. Element 7 is a size-limiting dielectric coating (PMMA or SiO₂). Alternatively, 8 shows pre-planarizing PMMA layer to allow flat surface for nanoimprinting or other uses. Element 6 is a vertical Au nanopillar (5-50 nm dia., or other metals, Pd, Pt, Ru, Rh, or island Au pad, or at least the pillar top surface region).

FIG. 1B(c) shows a MoS₂-based, two electrode or gated molecular sensor for genome or DNA sequencing 15, via detection of change of current pulse upon attaching of nucleotide, other biomolecules (or protein sensing). The MoS₂-based, two electrode or gated molecular sensor for genome or DNA sequencing 15 is shown to include an optional blocking agent 13 (e.g., PEG, Teflon, etc.) to prevent biomolecule adhesion, and a vertical Au nanopillar 14. The MoS₂-based, two electrode or gated molecular sensor for genome or DNA sequencing 15 is shown to include a single enzyme molecule 9 (e.g., DNA or RNA polymerase, or other sensors) attached onto a size limited MoS₂ bridge, with a biotin-streptavidin complex 10 (with other connecting moiety, e.g., silane). Element 11 is double strand DNA. Nucleotide monomer 12 (e.g., A, T, C, G) to be detected on polymerase reaction.

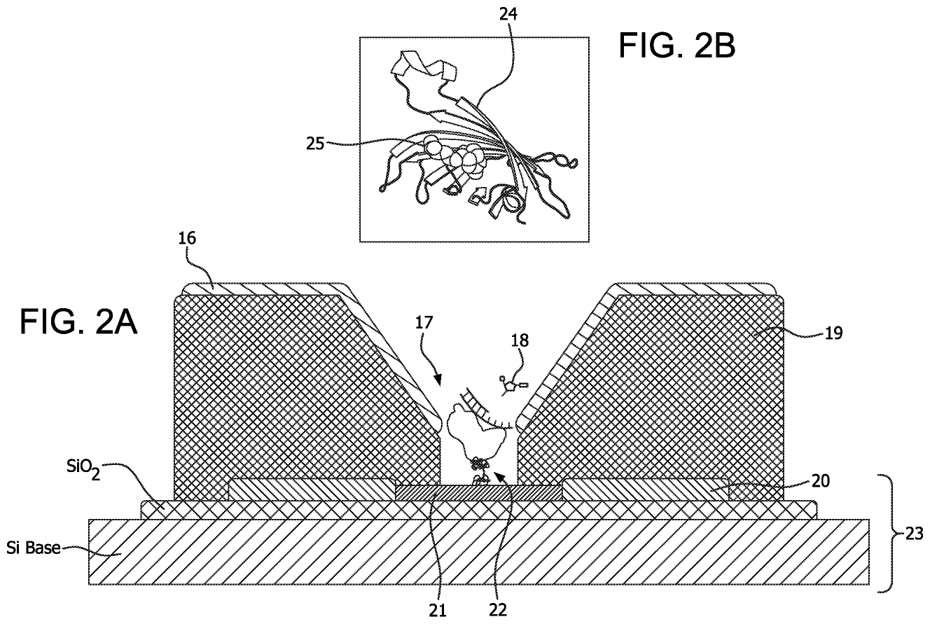

FIGS. 2A and 2B show Design #2 architecture for “Geometrically-guided, single DNA polymerase” molecular sensor on MoS₂ bridge (exposed size confined to −5 nm dia., comparable to streptavidin or polymerase size). FIG. 2A shows a FET sensor 23 based on MoS₂, WΩ type nano-ribbon bridge for genome or DNA sequencing via detection of change of current pulse upon attaching of nucleotide or other biomolecules. The structure 23 includes a PMMA, HSQ or silica based funnel type 19 (guiding structure), which is a tapered channel prepared by e-beam or microfabrication, or by nanoimprint mold (Si or metal). The structure 23 includes deposited metal electrode pair 20 (Au, Pd, Pt, Ru, etc.) for source, drain, with an optional third electrode as a gate structure, optional blocking agent 16 (e.g., PEG, Teflon) to prevent unwanted biomolecule adhesion), MoS₂ nano-ribbon bridge 21, a single polymerase 17 attached onto a size-limited MoS₂ nano-ribbon bridge, and biotin (on a semiconductor nano-ribbon surface, optionally with silane linker)+streptavidin (on polymerase)> or <antibody-antigen> linkage, or other binding mechanism 22. Nucleotide monomer 18 (e.g., A, T, C, G, etc.), fragments, or proteins to be detected on polymerase reaction. FIG. 2B shows streptavidin 24+biotin 25 binding mechanism.

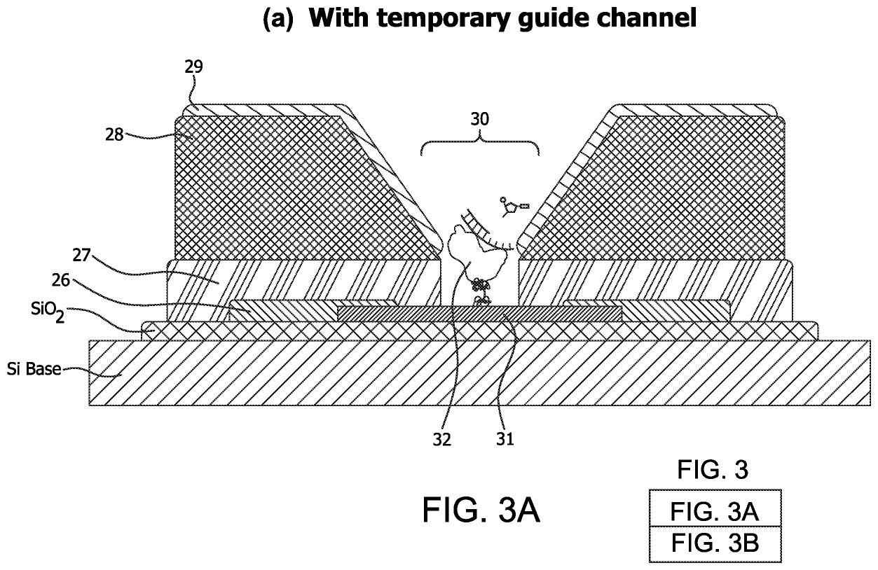

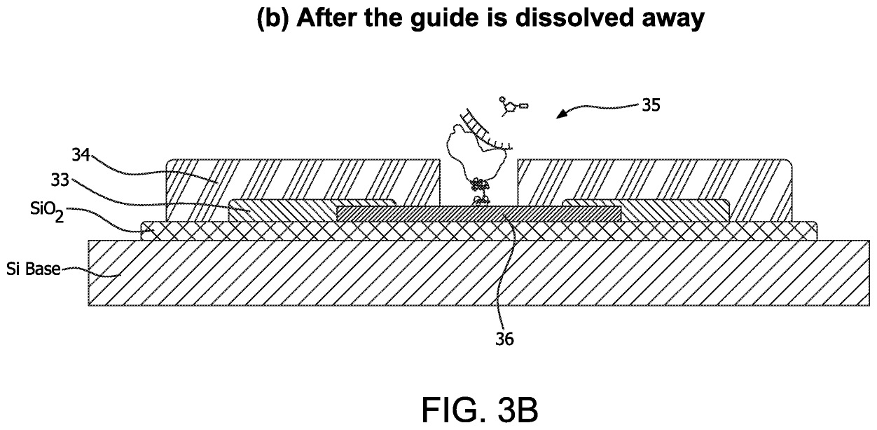

FIGS. 3A and 3B show Design #3 architecture for “Temporary guiding channel to construct single DNA polymerase” molecular sensor on MoS₂ bridge (exposed size confined to −5 nm dia., comparable to streptavidin or polymerase size). FIG. 3A(a) shows a structure with a temporary guide channel (guidance channel 30). The structure includes a temporary removable (sacrificial) layer 28 (dextrin, polysaccharide, polyvinyl alcohol, etc.) that can be dissolved in water, alcohol or some other solvent, or dissolvable resist such as PMMA, with optional blocking agent 29 (e.g., PEG, Teflon); an insoluble dielectric 27; deposited metal electrode pair 26 (Au, Pd, Pt, Ru, etc.) for source, drain, with an optional third electrode as a gate electrode; and MoS₂ nano-ribbon bridge 31. A single polymerase 32 is on MoS₂ bridge.

FIG. 3B(b) shows a structure after the guide is dissolved away. The structure includes insoluble (undissolvable) dielectric 34 such as SiO₂, cured PMMA, HSQ, etc., deposited metal electrode 33 (Au, Pd, Pt, Ru, etc.) and MoS₂ nano-ribbon bridge 36. The structure includes a single molecule polymerase sensor 35 partially hidden below the dielectric layer 34 (PMMA, SiO₂, etc.).

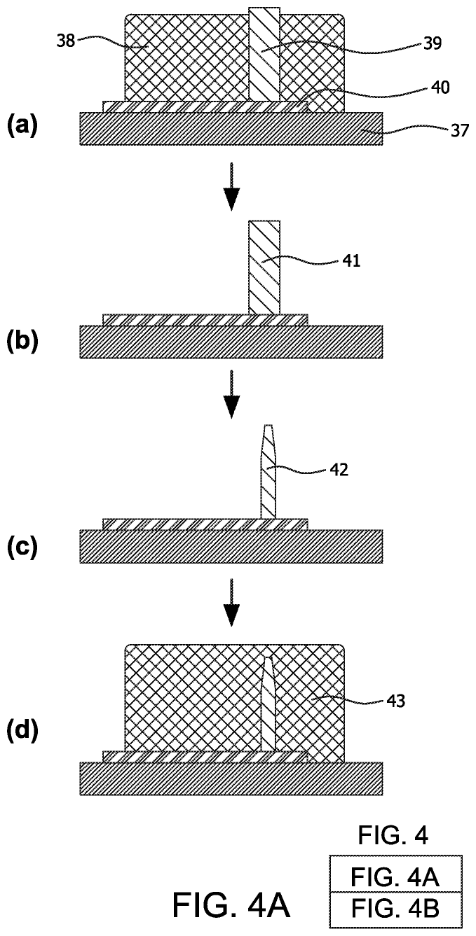

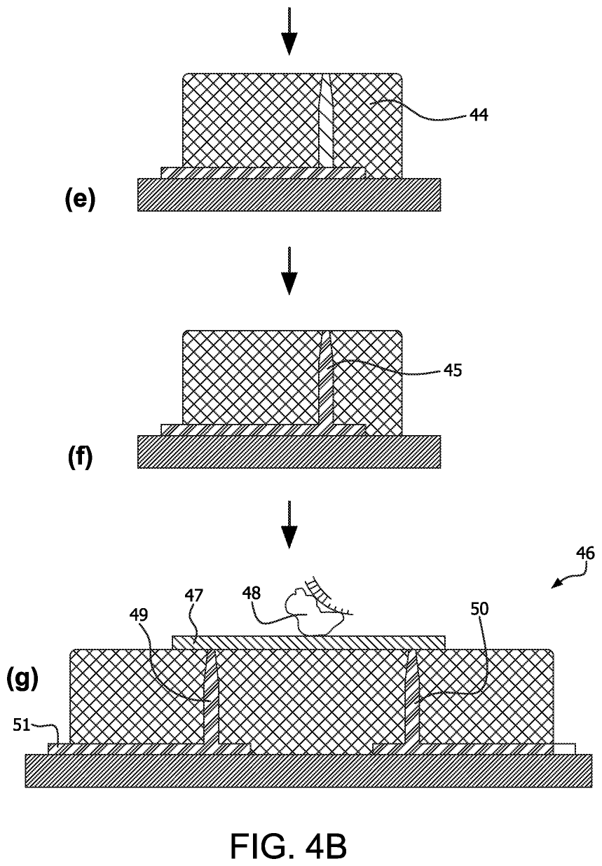

FIGS. 4A and 4B show a method. In FIG. 4A(a), the structure includes Au lead wire 40 on substrate 37. E-beam or nanoimprint lithography is used to make a vertical hole in PMMA 38, which is filled with Cu or Ni by electroless or electrodeposition, or (sputter+lift-off) to make a Cu nanopillar 39. In FIG. 4A(b), dissolve away PMMA to expose bare Cu nanopillar 41 (20-50 nm dia.). In FIG. 4A(c), chemical or RIE etch Cu nanowire 42 to reduce the diameter and make the tip pointed (e.g., ˜5 nm). In FIG. 4A(d), PMMA 43 is cast to cover the diameter-reduced Cu nanopillar. In FIG. 4B(e), PMMA 44 is planarized in height by RIE to expose the Cu nanopillar top. In FIG. 4B(f), dissolve away Cu nanopillar and electrodeposit Au 45 (only ˜5 nm dia. tip area) which can size-limit the MoS₂ width or the number of any attachable nanowire/nano-ribbon bridge. FIG. 4B(g) shows the molecular bridge solid state sensor 46 via detection of change of current pulse or other signals upon attaching or detaching of nucleotide or other biomolecules. Molecular bridge solid state sensor 46 includes: Au lead wire 51 on substrate; Au nanopillar 49; Au nanopillar 50; MoS₂ bridge FET 47; and single enzyme DNA polymerase 48 attached onto the MoS₂ bridge 47.

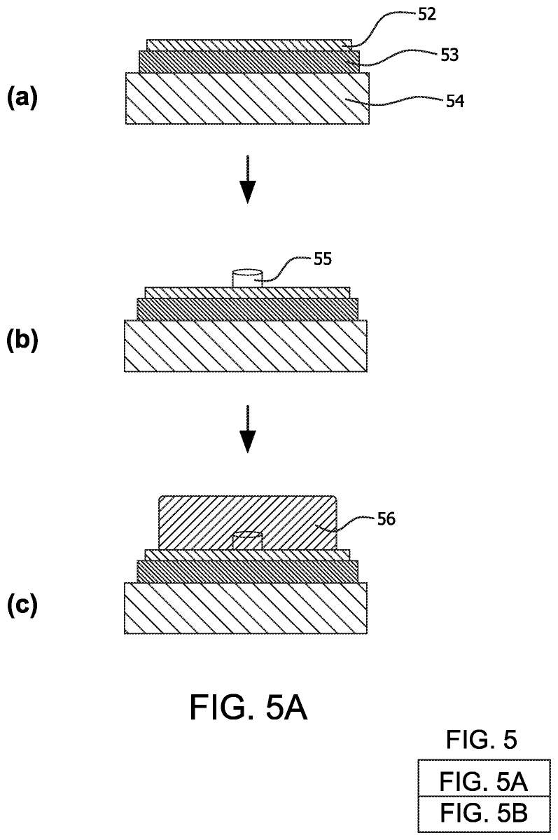

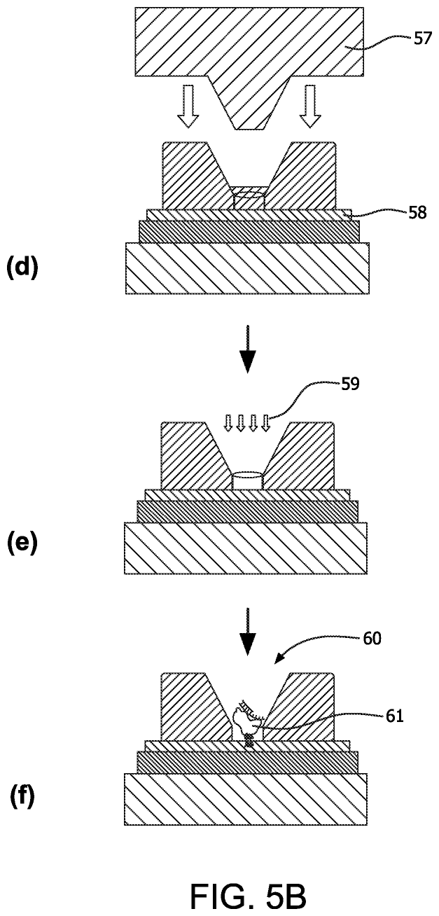

FIGS. 5A and 5B show a method. In FIG. 5A(a), the structure includes a MoS₂ nano-ribbon 52 on an underlayer 53 (e.g., PMMA, SiO₂), on a substrate 54. In FIG. 5A(b), a dissolvable pillar 55 (5-10 nm dia. Cu, Ni, etc.) is deposited by e-beam or NIL lithography. In FIG. 5A(c), a resist 56 (e.g., PMMA, HSQ, or other silica-containing resist) is spin coated. In FIG. 5B(d), an imprint is made with a die 57 (e.g., made of Si, SiO₂, metal alloy mold). The MoS₂ nano-ribbon 58 remains throughout. In FIG. 5B(e), the metal pillar is exposed by RIE etching 59. In FIG. 5B(f), the metal pillar is dissolved away to create a funnel shaped guiding structure 60 with e.g., 5-10 nm hole, so that only a single streptavidin or only a single molecule polymerase can be placed for more accurate sequencing. The structure in FIG. 5B(f) includes a single polymerase 61 attached onto a size-limited MoS₂ bridge (by e.g., biotin-streptavidin or other linkage).

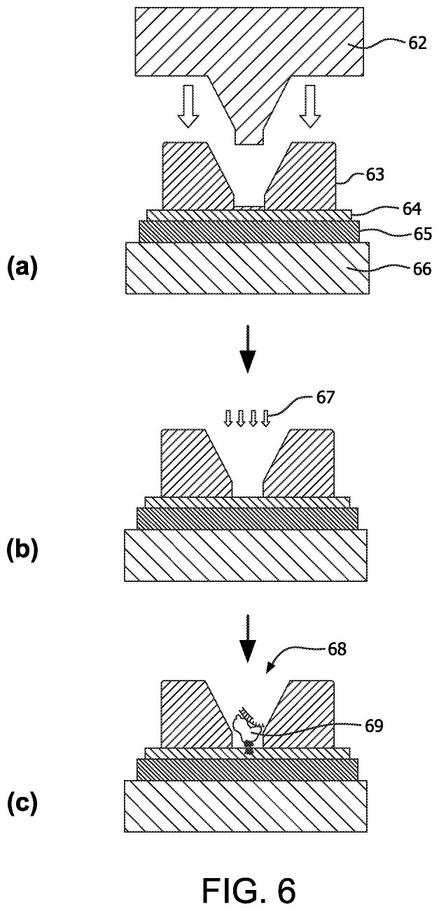

FIG. 6 shows a method. In FIG. 6(a), the structure includes a MoS₂ nano-ribbon 64 on a PMMA layer 65, which is on a Si substrate 66. A PMMA or HSQ (can be converted to SiO₂ later) thermo-plastic resist or other silica-containing resist 63 is imprinted with a die 62 (e.g., made of Si, SiO₂, metal alloy mold) having a <10 nm dia. tip protrusion. In FIG. 6(b), RIE etch 67 exposes the base MoS₂ bridge nano-ribbon surface. In FIG. 6(c), through the funnel shaped guiding structure 68 with e.g., 5-10 nm hole is placed only a single streptavidin or only a single molecule polymerase for more accurate sequencing. FIG. 6(c) shows a single polymerase 69 attached onto a size-limited MoS₂ bridge (by e.g., biotin-streptavidin or other linkage).

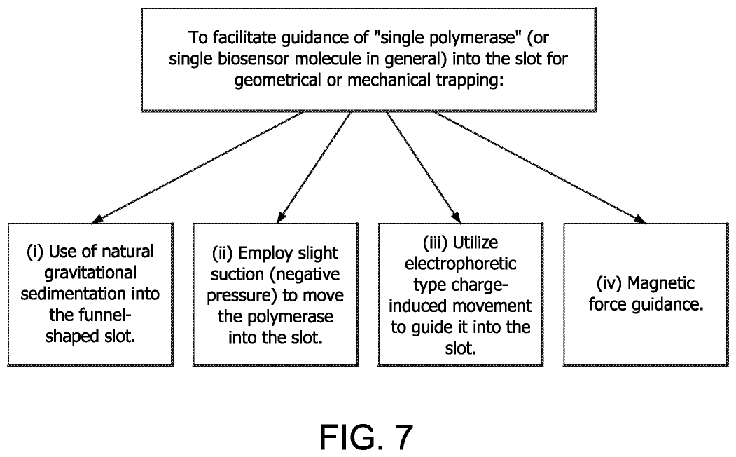

FIG. 7 shows how to facilitate guidance of “single polymerase” (or ingle biosensor molecule in general) into the slot for geometrical or mechanical trapping: (i) use of natural gravitational sedimentation into the funnel-shaped slot; (ii) employ slight suction (negative pressure) to move the polymerase into the slot; (iii) utilize electrophoretic type charge-induced movement to guide it into the slot; (iv) magnetic force guidance.

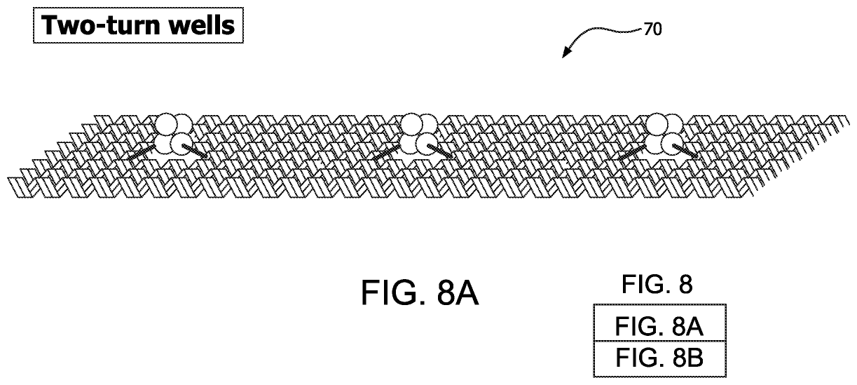

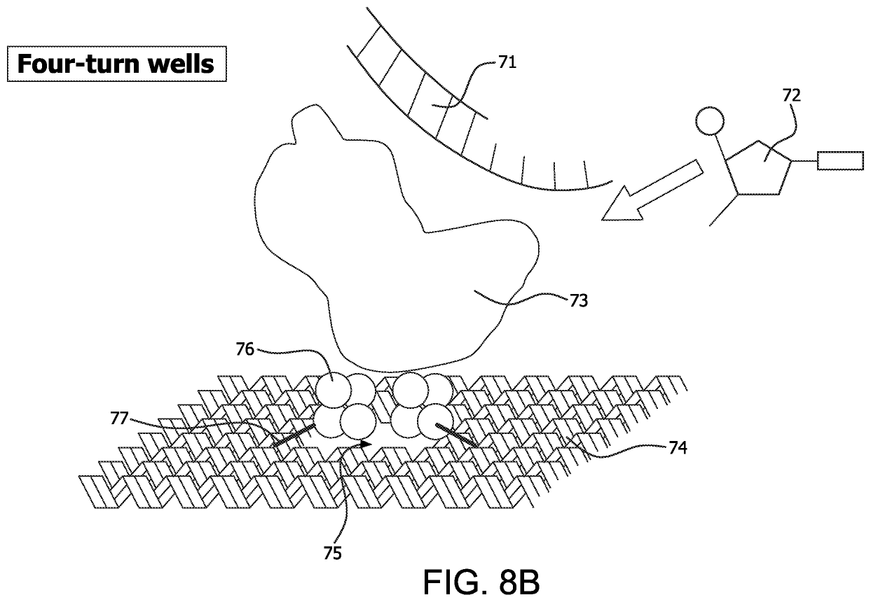

FIGS. 8A and 8B show Design #4 “Use of size-confined DNA assembly well”: size-confined nanowell by DNA assembly on MoS₂ nano-ribbon bridge; periodic array of Streptavidin (SA) on electrode surface, or such a template further processed/coated (with dielectric or conductive layer) for geometrical size-limiting constraint to enable only (or essentially only) a single enzyme molecule attachment; SA nanoarray is prepared by size-selective capturing of a single SA tetramer in a two-turn well (FIG. 8A, 70) or capturing of two tetramers trapped in a four-turn well (FIG. 8B). In FIG. 8A, two-turn wells 70 (20 nm-10 μm) spaced apart to be used for each electrode pair in multi-sensor array. In FIG. 8B, a four-turn well 75 (˜7×10 nm well) in DNA bundle 74 has trapped streptavidin 76 (two streptavidin tetramers), biotin 77 linking. FIG. 8B shows double strand DNA 71, nucleotide monomer 72 (A, T, C, G, etc.), short fragments, proteins to be detected, and preferably a single enzyme molecule 73 (e.g., DNA or RNA polymerase) attached onto size limited TMD bridge like MoS₂ or WS₂.

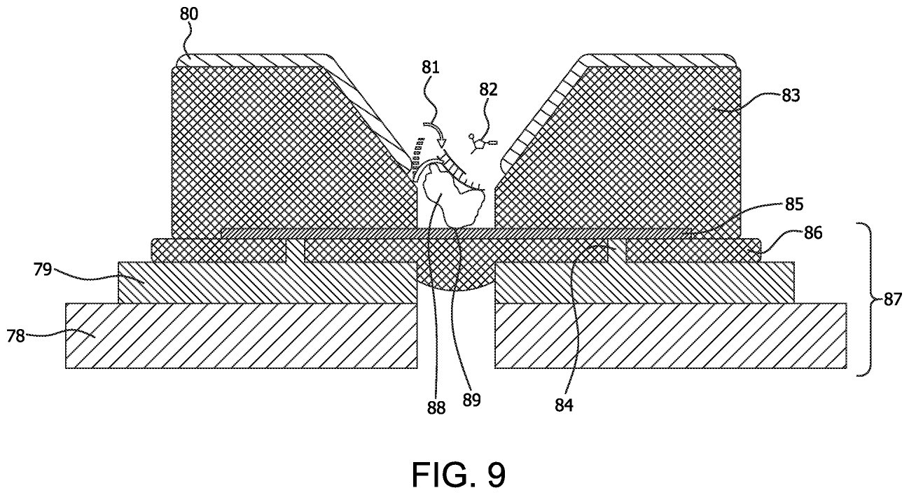

FIG. 9 shows Design #5 “Use of physically-trapped single polymerase on MoS₂ bridge on vertical nanopillar conductor,” to reduce contact resistance and further increase the FET signals (perhaps by >×10), eliminate biotin, streptavidin type connecting molecules; use of shape memory polymer or alloy structure to physically force electrical contacts (Van der Waals force bond) of polymerase molecule onto MoS₂ bridge. In FIG. 9, the molecular sensor on MoS₂ bridge 87, for genome or DNA sequencing via detection of change of current pulse upon attaching of nucleotide or other biomolecules, includes Au lead 79 on dielectric substrate 78 (SiO₂, etc., on Si); vertical Au nanopillar connect 84; planarizing PMMA 86 also layer to enable vertical Au connect 84; MoS₂ bridge 85, and PMMA 83. In FIG. 9, blocking agent 80 (e.g., PEG, Teflon) to prevent biomolecule adhesion. Shape memory change 81 to fix the polymerase, a single polymerase 88 attached onto a size-limited MoS₂ bridge, with nucleotide monomer 82 (e.g., A, T, C, G, U, etc.) to be detected on polymerase reaction. In FIG. 9, 89 is <Biotin (on MoS₂)+Streptavidin (on polymerase)>, or <antibody-antigen> linkage, or other mechanisms.

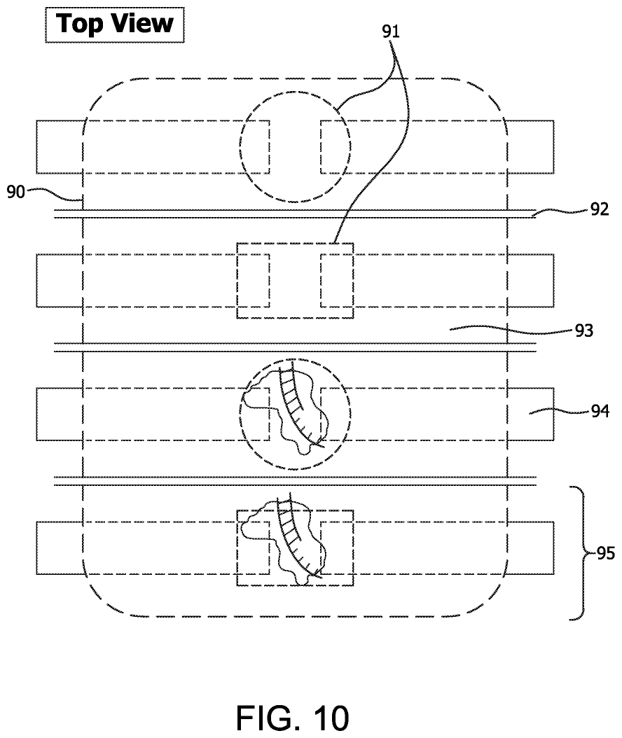

With reference to FIG. 10, a MoS₂ sheet 90 includes size-limited (circular, square or other shape) and locally selectively exposed TMD 91 like MoS₂. A large sheet can be placed by lifting up of floating sheet onto device surface covering many electrode pairs lithography-patterned, FIB separated, or mask patterned. Optional FIB (focused ion beam) slicing, e-beam/NIL slicing 92 of MoS₂ for separation from adjacent devices. The exposed island region is surrounded by pattern defined dielectric coating mask 93 (e.g., PMMA, PDMS, other adhesion blocking PEG type layer or Teflon, etc.) to prevent/minimize biomolecule attachment. An array of conducting electrode and lead wires 94 (Au, Pt, Ag, Pd, Rh, or their alloys, etc.) for signal detection. In FIG. 10, one unit of molecular bridge sensor 95 comprising a MoS₂ or WΩ layer bridge is shown.

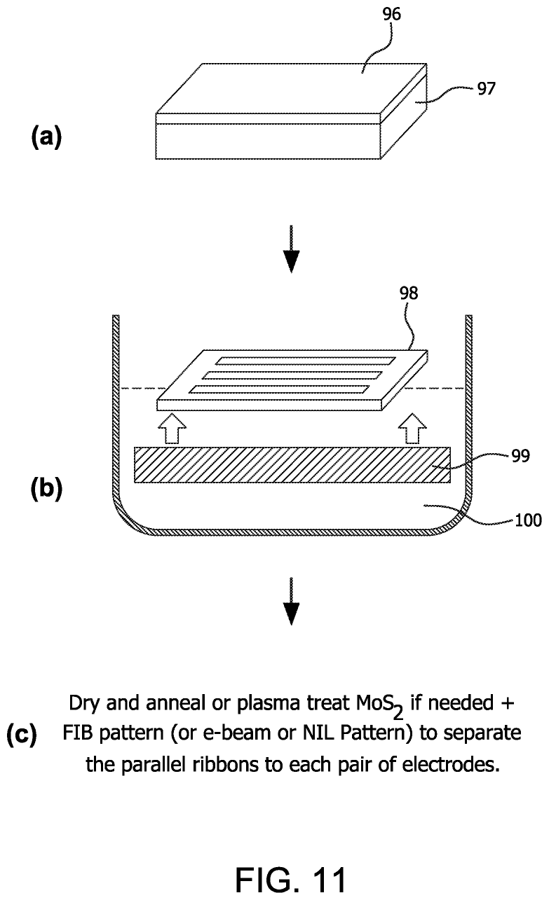

FIG. 11 shows “Wet transfer of unpatterned or pre-patterned MoS₂ ribbon array: MoS₂ films are synthesized at >700° C., so they have to be pre-made and then transferred onto sequencing device; Released in aqueous solution (chemically dissolve away the substrate); Place the Au electrode device (wafer) and lift up to catch the floating MoS₂ film; Dry and anneal to strongly bond the MoS₂ film (or nano-ribbon) on to Au electrode surface; Prepare size-limited MoS₂ regions and attach DNA polymerase; Perform nucleotide attachment and obtain sequence signals. In FIG. 11(a), synthesis of TMD 96 like MoS₂ or WΩ (e.g., by exfoliation, CVD or sulfurization on Si or metal substrate 97. Substrate 97 is Si with SiO₂, SOI wafer or metal layer. In the transition from (a) to (b), substrate etched away for floating MoS₂. In FIG. 11(b), floating MoS₂ or WΩ 98 (no pattern or pre-pattered into parallel ribbons, still attached) in H₂O or alcohol 99, with SiO₂-coated Si substrate 100 (optionally with Au electrode pair on it), lifted up to pick up MoS₂ layer. In FIG. 11(c), dry and anneal or plasma treat MoS₂ if needed +FIB pattern (or e-beam or NIL pattern) to separate the parallel ribbons to each pair of electrodes.

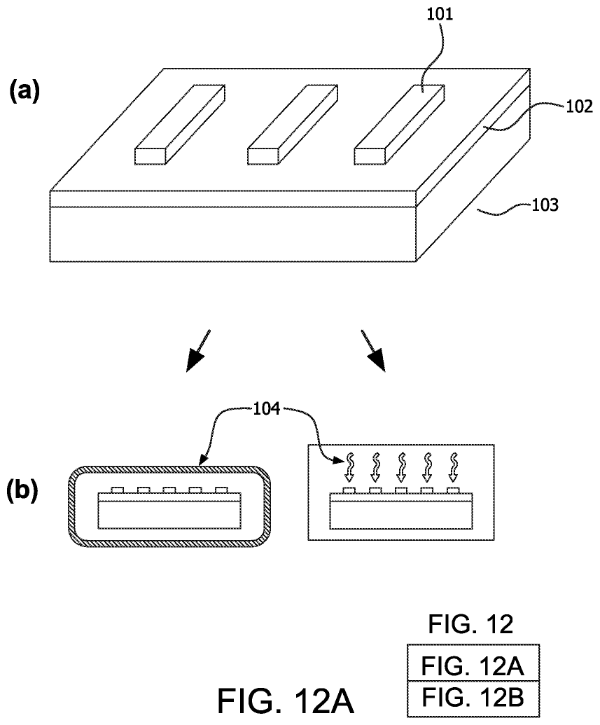

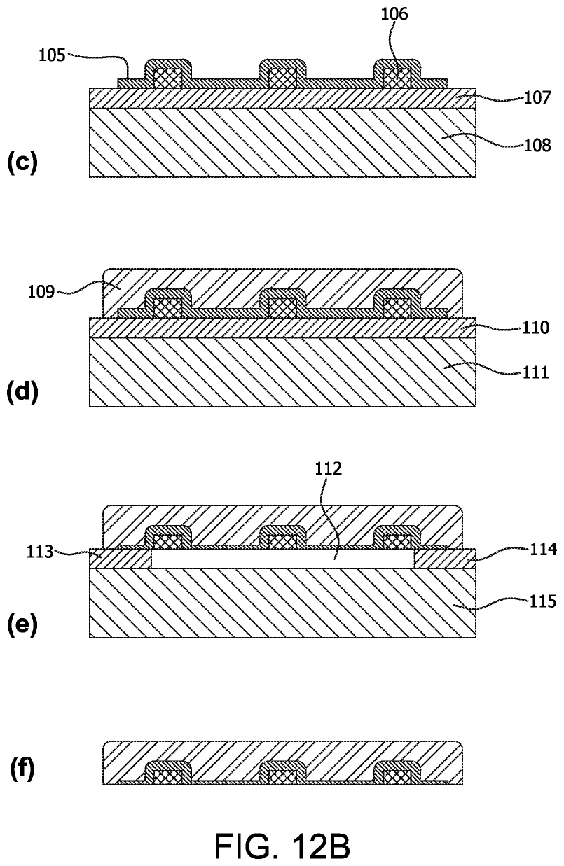

FIGS. 12A and 12B show a method. The structure in FIG. 12A(a) includes nano-patterned MoS₂ nano-ribbon array 101 on SiO₂ 102 on Si base 103. In FIG. 12A(b), high-temp anneal or plasma treat 104 to repair possible damages on patterning. In FIG. 12AB(c), nano-patterned MoS₂ nano-ribbon array 106 on SiO₂ 107 on Si base 108 is optionally coated with removable polymer 105 (e.g., dextrin, glucose, grease, wax). In FIG. 12B(d), nano-patterned MoS₂ nano-ribbon array on SiO₂ 110 on Si base 111, optionally coated with removable polymer (e.g., dextrin, glucose, grease, wax) is cast with PMMA or PDMS elastomer 109 and cure. In FIG. 12B(e), the SiO₂ 112 underneath is etched away through intentionally added slots in PMMA or PDMS to release the potted nano-ribbon array. This can also be done in two-steps of PMMA followed by PDMS. In FIG. 12B(e), the structure includes portions of SiO₂ 113 and 114 after etching, on Si base 115. FIG. 12B(f) shows the released material that can be washed lightly to make the MoS₂ nano-ribbons protrude slightly, dried, and then transferred and pressed onto the sequencing device to be released, and form a non-bridge between two mating metallic electrodes.

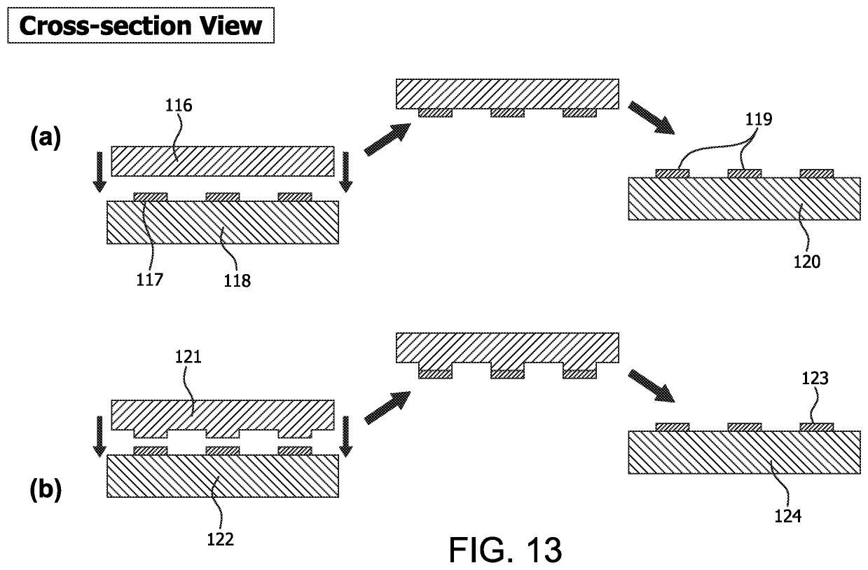

FIG. 13 shows a method. FIG. 13(a) shows a PDMS stamp 116 (flat), and MoS₂ nano-ribbon array 117 (nanopatterned on a flat substrate 118). The transformation illustrate is pick up the nanoribbons by stamp, and release the nano-ribbons 119 on device surface 120 (e.g., as a bridge between two mating electrodes). In FIG. 13(b) PDMS stamp (pre-shaped) 121 is used to pick up the nanoribbons from substrate 122 by stamp, the nano-ribbons 123 released on device surface 124 (e.g., as a bridge between two mating electrodes).

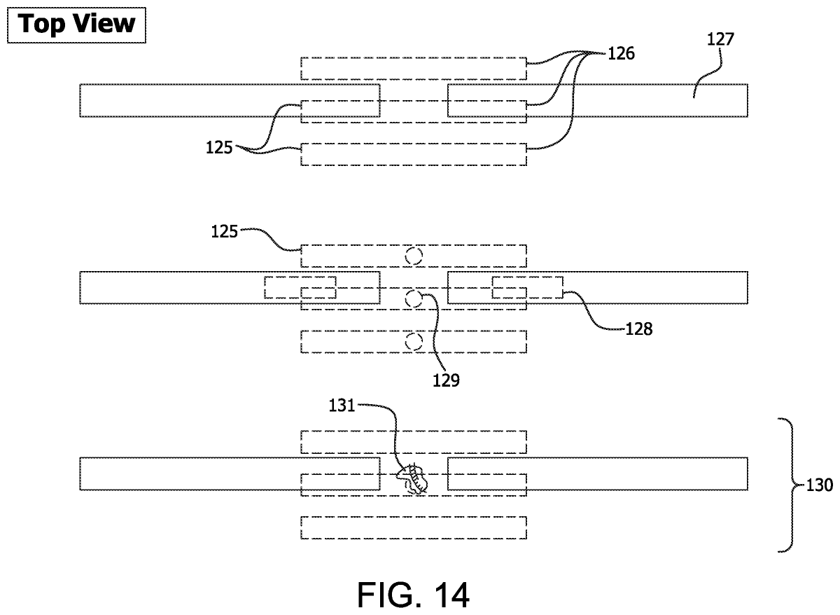

FIG. 14 shows MoS₂ nano-ribbons 125 in a pre-patterned parallel array 126 of MoS₂ with redundant ribbon array (made by e.g., e-beam lithography, nanoimprint lithography, template-assisted nanopatterning, etc.). Imprint transferred onto Au electrode array so as to increase the probability of MoS₂ bridge connection. FIG. 14 shows an array of conducting electrode/lead wires 127 (Au, Pt, Ag, Pd, Rh, etc.) for signal detection. The MoS₂ ribbons can be Van der Waals force attached, or optionally metallization deposit (metallization deposit 128 (Ti, Au, Ni, etc.) over MoS₂) can be made to more firmly attach the ribbon on the electrode. In FIG. 14, optional addition of local biotin-binding-enhancing coating 129, or surrounding-area-masking to limit exposed MoS₂ region to −5-10 nm dia. One unit of molecular sensor 130 comprises a MoS₂ bridge with polymerase assembly, shown with enzyme polymerase 131 (with associated biotin-streptavidin, etc.).

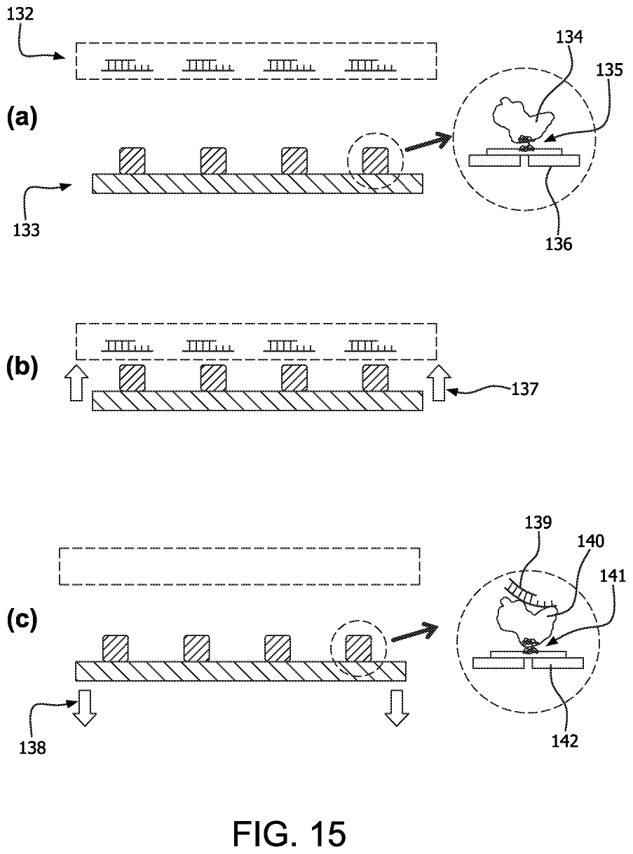

FIG. 15 shows a method. In FIG. 15(a), a tethered array of encoded (memory written) DNA fragments 132 periodically positioned on a substrate. Molecular nanobridge (MoS₂) polymerase sensor array 133 (w/o DNA template). Magnified view shows polymerase 134, linker 135, and Au electrode pair 136. In FIG. 15(b), polymerase-MoS₂ nanobridge array approaching DNA array being released (indicated by 137). In FIG. 15(c), nanobridge sensor array polymerase molecule array picks up DNA templates and moved away (indicated by 138). Magnified view shows DNA template 139, polymerase 140, linker 141 and Au electrode 142.

1. A single-molecule biosensor comprising: a conducting electrode pair disposed on a substrate, the electrode pair comprising a first electrode and a second electrode spaced-apart from the first electrode by a nanogap; a contiguous transition metal dichalcogenide (TMD) layer disposed on the electrode and on the second electrode, the TMD layer configured as a bridge suspended over the nanogap; an enzyme molecule attached to a region of the TMD layer suspended over the nanogap; and a microfluidic system encasing the conducting electrode pair, the contiguous TMD layer and the enzyme molecule attached thereto.

2. The single-molecule biosensor of claim 1, wherein the TMD layer comprises a TMD selected from the group consisting of MoS₂, WS₂, TiS₂, ZrS₂, HfS₂, VS₂, NbS₂, TaS₂, TcS₂, ReS₂, CoS₂, RhS₂, IrS₂, NiS₂, PdS₂, PtS₂, and mixtures thereof.

3. The single-molecule biosensor of claim 1, wherein the TMD layer comprises at least one TMD of structure MS₍₂₋ₓ₎ or MS₍₂₊ₓ₎, wherein x is 0-0.3, and M is Mo, W, Ti, Zr, Hf, V, Nb, Ta, Tc, Re, Co, Rh, Ir, Ni, Pd, or Pt.

4. The single-molecule biosensor of claim 1, wherein the TMD layer comprises a TMD selected from the group consisting of MoSe₂, WSe₂, TiSe₂, ZrSe₂, HfSe₂, VSe₂, NbSe₂, TaSe₂, TcSe₂, ReSe₂, CoSe₂, RhSe₂, IrSe₂, NiSe₂, PdSe₂, PtSe₂, and mixtures thereof.

5. The single-molecule biosensor of claim 1, wherein the TMD layer comprises at least one TMD of structure MSe₍₂₋ₓ₎ or MSe₍₂₊ₓ₎, wherein x is 0-0.3, and M is Mo, W, Ti, Zr, Hf, V, Nb, Ta, Tc, Re, Co, Rh, Ir, Ni, Pd, or Pt.

6. The single-molecule biosensor of claim 1, wherein the TMD layer comprises a TMD selected from the group consisting of MoTe₂, WTe₂, TiTe₂, ZrTe₂, Hffe₂, VTe₂, NbTe₂, TaTe₂, TcTe₂, ReTe₂, CoTe₂, RhTe₂, IrTe₂, NiTe₂, PdTe₂, PtTe₂, and mixtures thereof.

7. The single-molecule biosensor of claim 1, wherein the TMD layer comprises at least one TMD of structure MTe₍₂₋ₓ₎ or MSe₍₂₊ₓ₎, wherein x is 0-0.3, and M is Mo, W, Ti, Zr, Hf, V, Nb, Ta, Tc, Re, Co, Rh, Ir, Ni, Pd, or Pt.

8. The single-molecule biosensor of claim 1, wherein the TMD layer comprises a mixed TMD compound selected from the group consisting of Mo(SₓSe_{y}Te_{z})₂, W(SₓSe_{y}Te_{z})₂, Ti(SₓSe_{y}Te_{z})₂, Zr(SₓSe_{y}Te_{z})₂, Hf(SₓSe_{y}Te_{z})₂, V(SₓSe_{y}Te_{z})₂, Nb(SₓSe_{y}Te_{z})₂, Ta(SₓSe_{y}Te_{z})₂, Tc(SₓSe_{y}Te_{z})₂, Re(SₓSe_{y}Te_{z})₂, Co(SₓSe_{y}Te_{z})₂, Rh(SₓSe_{y}Te_{z})₂, Ir(SₓSe_{y}Te_{z})₂, Ni(SₓSe_{y}Te_{z})₂, Pd(SₓSe_{y}Te_{z})₂, Pt(SₓSe_{y}Te_{z})₂, and mixtures thereof, wherein (x+y+z) is 0.7-1.3.

9. The single-molecule biosensor of claim 1, wherein the TMD layer comprises at least one TMD compound of structure M_{(1-w)}N_{y}X_{(2-z)}Y_{z}, wherein M is Al, Si, Ga, Ge, In, Sn, Sb, Bi, Na, K, Ca, Mg, Sr, or Ba; X is S, Se, or Te; Y is Li, B, C, N, O, P, F, Cl, or I; w is 0-0.3; and z is 0-0.3.

10. The single-molecule biosensor of claim 1, wherein the substrate comprises SiO₂ or Al₂O₃.

11. The single-molecule biosensor of claim 1, wherein the pair of conducting electrodes comprise at least one of Au, Pt, Ag, Pd, Rh, Ru, or alloys thereof.

12. The single-molecule biosensor of claim 1, wherein the nanogap is from about 1 nm to about 50 nm.

13. The single-molecule biosensor of claim 1, further comprising a dielectric, ceramic or polymer coating layer disposed on the TMD layer on a side opposite the first and second electrodes, wherein the dielectric, ceramic or polymer coating layer includes an opening on the region of the TMD layer suspended over the nanogap, the opening leaving an exposed portion of the TMD layer therein, the opening dimensioned to accommodate only one enzyme molecule.

14. The single-molecule biosensor of claim 13, wherein the dielectric, ceramic or polymer layer comprises PMMA or SiO₂.

15. The single-molecule biosensor of claim 1, wherein the enzyme molecule comprises a DNA polymerase or an RNA polymerase.

16. The single-molecule biosensor of claim 1, wherein the enzyme molecule is attached to the TMD layer via a biotin-streptavidin linkage.

17. The single-molecule biosensor of claim 1, wherein the TMD layer includes as least one of vacancy defects, interstitial defects, and aggregated defects.

18. The single-molecule biosensor of claim 1, wherein the pair of conducting electrodes are configured in a circuit as source and drain electrodes, and wherein the circuit further comprises a gate electrode disposed under the nanogap.

19. A DNA or RNA sequencing device comprising:
an electrode array of conducting electrode pairs disposed on a substrate, each pair of electrodes comprising a source electrode and a drain electrode spaced-apart from the source electrode by a nanogap;
a contiguous transition metal dichalcogenide (TMD) layer disposed on the first electrode and on the second electrode, the TMD layer configured as a bridge suspended over the nanogap;
a polymerase enzyme molecule attached to a region of the TMD layer over the nanogap; and
a microfluidic system encasing the conducting electrode pair, the contiguous TMD layer and the enzyme molecule attached thereto.

20. The DNA or RNA sequencing device of claim 19, wherein the TMD layer comprises at least one TMD of structure MX₍₂₋ₓ₎ or MX₍₂₊ₓ₎, wherein X is S, Se or Te; x is 0-0.3; and M is Mo, W, Ti, Zr, Hf, V, Nb, Ta, Tc, Re, Co, Rh, Ir, Ni, Pd, or Pt.
